(19) United States
(12) Patent Application Publication
(10) Pub. No.: US 2010/0310678 A1
(43) Pub. Date: Dec. 9, 2010

(54) Compositions and Methods for Nutrition Supplementation

(76) Inventor: John A. Giordano, West Orange, NJ (US)

Correspondence Address: DON J. PELTO;Sheppard, Mullin, Richter & Hampton LLP, 1300 I STREET, NW, 11TH FLOOR EAST, WASHINGTON, DC, 20005, US

(21) Appl. No.: 12/796,785

(22) Filed: Jun. 9, 2010

Related U.S. Application Data
(63) Continuation of application No. 10/901,054, filed on Jul. 29, 2004, now abandoned.

(51) Int. Cl. A61K 33/10; A61P 9/00

(52) U.S. Cl. 424/687

(57) ABSTRACT

The present invention relates to compositions, that may be swallowable, chewable or dissolvable, comprising various vitamins and minerals, and in a specific embodiment, comprise vitamin B₆, vitamin B₉, vitamin B₁₂, calcium, vitamin D₃, magnesium, and boron, and methods for using these compositions for nutritional supplementation in order to prevent, treat and/or alleviate the occurrence or negative effects of cardiovascular disease, colorectal cancer and osteoporosis.

### CROSS REFERENCE TO RELATED APPLICATIONS

The Present application is a Continuation and claims the benefit, under 35 U.S.C. §120, of U.S. patent application Ser. No. 10/901,054 filed Jul. 29, 2004, which is expressly incorporated fully herein by reference.

### FIELD OF THE INVENTION

The present invention relates to compositions, that may be swallowable, chewable and/or dissolvable, comprising various vitamins and minerals, and methods for using these compositions for nutritional supplementation and in order to prevent, treat and/or alleviate the occurrence or negative effects of cardiovascular disease, colorectal cancer and/or osteoporosis.

### BACKGROUND OF THE INVENTION

Cardiovascular disease continues to be the number one cause of death for both men and women in the United States. Statistics Related to Heart Disease, available at www.health.uab.edu/show.asp?durki=39661 (last visited 22 May 2004). Colorectal cancer is the second leading cause of death from cancer in the United States, claiming approximately 55,000 lives each year. Colorectal Cancer FactSheet, available at www.fdhn.org/html/education/colorectal/facts.html (last visited 22 May 2004). Osteoporosis, or a loss of bone mass and density, also is a major health concern. An estimated 28 million Americans presently have some form of osteoporosis—of these, greater than 80% are female. NIH Consensus Development Panel, J. AMER. MED. ASSOC. 785-95 (2001). Recent developments in nutritional research suggest that nutritional supplementation with specific vitamins and minerals, as an adjunct to proper diet, exercise and medical care, can aid in preventing, treating and/or alleviating the occurrence or negative effects of these diseases.

Nutrition plays a critical role in maintaining good health, and nutritional supplementation serves a vital role in protecting against poor nutrition and disease. For example, recent research has shown that vitamins and minerals help to prevent, treat and/or alleviate the occurrence or negative effects of cardiovascular disease, colorectal cancer and osteoporosis. While supplementation with certain vitamins and minerals protects against the onset of these diseases, other vitamins and minerals have been found to inhibit the beneficial effects of these certain vitamins and minerals. Specifically, B-complex vitamins, such as vitamins B₆, B₉ and B₁₂, calcium, vitamin D₃, magnesium and boron play integral roles in physiological mechanisms that serve to prevent, treat and/or alleviate the occurrence or negative effects of cardiovascular disease, colorectal cancer and osteoporosis. Supplementation with vitamins and minerals such as vitamin A, vitamin K, and iron may inhibit the beneficial effects of the B-complex vitamins, calcium, vitamin D₃, magnesium and boron. Thus, when creating or choosing a nutritional supplement, it is essential to understand the physiological needs and risks of individual patients and population groups and the interactions between various vitamins and minerals.

Further, while some patients may prefer swallowable dosage forms, it is estimated that 50% of the population has problems swallowing whole tablets. Seager, 50 J. PHARM. PHARMACOL. 375-82 (1998). These problems can lead to poor compliance, or even noncompliance, with dosing regimens and thus have a negative impact on prevention treatment efficiency. Id. Administration of vitamins and minerals through chewable or dissolvable compositions solves this problem because the compositions need not be swallowed whole.

### SUMMARY OF THE INVENTION

The present invention provides compositions and methods of using the compositions for both prophylactic and therapeutic nutritional supplementation. Specifically, the present invention includes vitamins and minerals that prevent, treat and/or alleviate the occurrence or negative effects of cardiovascular disease, colorectal cancer and osteoporosis. The present invention also may be formulated to exclude vitamins and minerals known to inhibit the beneficial effects of the included vitamins and minerals.

The compositions of the present invention may be in a swallowable, chewable or dissolvable form according to an individual patient's preferences. Choice in dosage form promotes ease of administration and compliance with dosing regimens.

In one embodiment of the present invention, the compositions may include one or more of B-complex vitamins, such as B₆, B₉ and B₁₂, calcium, vitamin D₃, magnesium, and boron.

In another embodiment of the present invention, the B-complex vitamins may include one or more of vitamin B₆ in the form of pyridoxine hydrochloride; vitamin B₉ in the form of folic acid; and/or vitamin B₁₂ in the form of cyanocobalamin. In another embodiment, the compositions and methods of the present invention may include vitamin B₉ in the form of folacin, metafolin, folate or natural isomers thereof including (6S)-tetrahydrofolic acid, 5-methyl-(6S)-tetrahydrofolic acid, 5-formyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid or polyglutamyl derivatives thereof.

In another embodiment of the present invention, the compositions may comprise one or more of vitamin B₆ in the form of pyridoxine hydrochloride; vitamin B₉ in the form of folic acid, vitamin B₁₂ in the form of cyanocobalamin; calcium in the form of calcium carbonate; vitamin D₃ in the form of cholecalciferol; magnesium in the form of magnesium oxide; and/or boron in the form of boron amino acid chelate.

In another embodiment of the present invention, the compositions may be substantially free of one or more of added vitamin A, added vitamin K, added iron, and added lactose.

In another embodiment, the compositions of the present invention may be substantially free of added beta carotene; substantially free of added alpha carotene; substantially free of added lutein; substantially free of added lycopene; substantially free of added zeaxanthin; substantially free of added vitamin B₁; substantially free of added vitamin B₂; substantially free of added vitamin B₃; substantially free of added vitamin B₄; substantially free of added vitamin B₅; substantially free of added vitamin B₆; substantially free of added vitamin B₇; substantially free of added vitamin B₈; substantially free of added vitamin B₉; substantially free of added vitamin B₁₀; substantially free of added vitamin B₁₁; substantially free of added vitamin B₁₂; substantially free of added vitamin C; substantially free of added vitamin D₃; substantially free of added vitamin E; substantially free of added calcium; substantially free of added chromium; substantially free of added copper; substantially free of added magnesium; substantially free of added manganese; substantially free of added selenium; substantially free of added zinc; substantially free of added boron; substantially free of added odorless garlic; substantially free of added coenzyme Q-10; substantially free of added 1-carnitine; substantially free of added grape seed extract; substantially free of added green tea extract; substantially free of added quercetin; substantially free of added hawthorne berries; and/or substantially free of added alpha lipoic acid.

In another embodiment, the compositions of the present invention may comprise pharmaceutically acceptable carriers, such as one or more of binders, diluents, lubricants, glidants, colorants, emulsifiers, disintegrants, starches, water, oils, alcohols, preservatives, and sugars.

In another embodiment of the present invention, the compositions may comprise sweetening agents such as one or more of sucrose, fructose, high fructose corn syrup, dextrose, saccharin sodium, maltodextrin, aspartame, potassium acesulfame, neohesperidin dihydrochalcone, sucralose, monoammonium glycyrrhizinate, and mixtures thereof.

In another embodiment of the present invention, the compositions may comprise flavorants such as one or more of anise oil, cinnamon oil, peppermint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, grape oil, apple essence, pear essence, peach essence, berry essence, wildberry essence, date essence, blueberry essence, kiwi essence, strawberry essence, raspberry essence, cherry essence, plum essence, pineapple essence, apricot essence, natural mixed berry flavor, citric acid, malic acid, vanilla, vanillin, cocoa, chocolate, and menthol.

In another embodiment of the present invention, the compositions may comprise alkyl polysiloxane in the form of dimethyl polysiloxane.

In another embodiment of the present invention, the compositions may be in a chewable chocolate form comprising fructose, chocolate, plasdone, isopropyl alcohol, acacia gum, natural chocolate flavor, stearic acid, silicon dioxide, and magnesium stearate.

In another embodiment, the compositions of the present invention may comprise one or more of about 5 mg to about 15 mg vitamin B₆; about 1 mg to about 3 mg folic acid; about 12 μg to about 38 μg vitamin B₁₂; about 250 mg to about 750 mg calcium; about 100 IU to about 300 IU vitamin D₃; about 25 mg to about 75 mg magnesium; and about 0.5 mg to about 2 mg boron.

In another embodiment, the compositions of the present invention may comprise one or more of about 8 mg to about 12 mg vitamin B₆; about 1 mg to about 2.2 mg folic acid; about 20 μg to about 30 μg vitamin B₁₂; about 400 mg to about 600 mg calcium; about 160 FU to about 240 IU vitamin D₃; about 40 mg to about 60 mg magnesium; and about 0.5 mg to about 1.5 mg boron.

In another embodiment, the compositions of the present invention may comprise one or more of about 9 mg to about 11 mg vitamin B₆; about 1.5 mg to about 1.75 mg folic acid; about 22 μg to about 28 μg vitamin B₁₂; about 450 mg to about 550 mg calcium; about 180 IU to about 220 IU vitamin D₃; about 45 mg to about 55 mg magnesium; and about 0.8 mg to about 1.2 mg boron.

In another embodiment, the compositions of the present invention may comprise one or more of about 10 mg vitamin B₆; about 1.6 mg folic acid; about 25 μg vitamin B₁₂; about 500 mg calcium; about 200 IU vitamin D₃; about 50 mg magnesium; and about 1 mg boron.

In another embodiment of the present invention, the compositions are administered to a patient to prevent, treat and/or alleviate the occurrence or negative effects of cardiovascular disease, colorectal cancer and osteoporosis.

The present invention also includes methods of administering the compositions of the invention to patients as a prophylactic measure to prevent, treat and/or alleviate the occurrence or negative effects of cardiovascular disease, colorectal cancer and osteoporosis.

In one embodiment of the present invention the methods may utilize compositions comprising B-complex vitamins, calcium, vitamin D₃, magnesium, and boron.

In another embodiment of the present invention, the methods may utilize compositions in a swallowable, chewable or dissolvable form.

In another embodiment of the present invention, the methods may utilize compositions including vitamin B₆ in the form of pyridoxine hydrochloride; vitamin B₉ in the form of folic acid and/or vitamin B₁₂ in the form of cyanocobalamin. In another embodiment of the present invention, vitamin B₉ may be in the form of one or more of folacin, metafolin, folate or natural isomers thereof including (6S)-tetrahydrofolic acid, 5-methyl-(6S)-tetrahydrofolic acid, 5-formyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid or polyglutamyl derivatives thereof.

In another embodiment of the present invention, the methods may utilize compositions comprising one or more of vitamin B₆ in the form of pyridoxine hydrochloride; vitamin B₉ in the form of folic acid, vitamin B₁₂ in the form of cyanocobalamin; calcium in the form of calcium carbonate; vitamin D₃ in the form of cholecalciferol; magnesium in the form of magnesium oxide; and boron in the form of boron amino acid chelate.

In another embodiment of the present invention, the methods may utilize compositions substantially free of one or more of added vitamin A, added vitamin K, added iron and added lactose.

In another embodiment, the methods of the present invention may utilize compositions substantially free of added beta carotene; substantially free of added alpha carotene; substantially free of added lutein; substantially free of added lycopene; substantially free of added zeaxanthin; substantially free of added vitamin B₁; substantially free of added vitamin B₂; substantially free of added vitamin B₃; substantially free of added vitamin B₄; substantially free of added vitamin B₅; substantially free of added vitamin B₆; substantially free of added vitamin B₇; substantially free of added vitamin B₈; substantially free of added vitamin B₉; substantially free of added vitamin B₁₀; substantially free of added vitamin B₁₁; substantially free of added vitamin B₁₂; substantially free of added vitamin C; substantially free of added vitamin D₃; substantially free of added vitamin E; substantially free of added calcium; substantially free of added chromium; substantially free of added copper; substantially free of added magnesium; substantially free of added manganese; substantially free of added selenium; substantially free of added zinc; substantially free of added boron; substantially free of added odorless garlic; substantially free of added coenzyme Q-10; substantially free of added 1-carnitine; substantially free of added grape seed extract; substantially free of added green tea extract; substantially free of added quercetin; substantially free of added hawthorne berries; and/or substantially free of added alpha lipoic acid.

In another embodiment of the present invention, the methods may utilize compositions comprising pharmaceutically acceptable carriers, such as one or more of binders, diluents, lubricants, glidants, colorants, emulsifiers, disintegrants, starches, water, oils, alcohols, preservatives and sugars.

In another embodiment of the present invention, the methods may utilize compositions comprising sweetening agents, such as one or more of sucrose, fructose, high fructose corn syrup, dextrose, saccharin sodium, maltodextrin, aspartame, potassium acesulfame, neohesperidin dihydrochalcone, sucralose, monoammonium glycyrrhizinate, and mixtures thereof.

In another embodiment of the present invention, the methods may utilize compositions comprising flavorants such as one or more of anise oil, cinnamon oil, peppermint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, grape oil, apple essence, pear essence, peach essence, berry essence, wildberry essence, date essence, blueberry essence, kiwi essence, strawberry essence, raspberry essence, cherry essence, plum essence, pineapple essence, apricot essence, natural mixed berry flavor, citric acid, malic acid, vanilla, vanillin, cocoa, chocolate, and menthol.

In another embodiment of the present invention, the methods may utilize compositions comprising alkyl polysiloxane in the form of dimethyl polysiloxane.

In another embodiment of the present invention, the methods may utilize compositions in a chewable chocolate form comprising fructose, chocolate, plasdone, isopropyl alcohol, acacia gum, natural chocolate flavor, stearic acid, silicon dioxide, and magnesium stearate.

In another embodiment, the methods may utilize compositions comprising one or more of about 5 mg to about 15 mg vitamin B₆; about 1 mg to about 3 mg folic acid; about 12 μg to about 38 μg vitamin B₁₂; about 250 mg to about 750 mg calcium; about 100 IU to about 300 IU vitamin D₃; about 25 mg to about 75 mg magnesium; and about 0.5 mg to about 2 mg boron.

In another embodiment of the present invention, the methods may utilize compositions comprising one or more of about 8 mg to about 12 mg vitamin B₆; about 1 mg to about 2.2 mg folic acid; about 20 μg to about 30 μg vitamin B₁₂; about 400 mg to about 600 mg calcium; about 160 IU to about 240 IU vitamin D₃; about 40 mg to about 60 mg magnesium and about 0.5 mg to about 1.5 mg boron.

In another embodiment of the present invention, the methods may utilize compositions comprising one or more of about 9 mg to about 11 mg vitamin B₆; about 1.5 mg to about 1.75 mg folic acid; about 22 μg to about 28 μg vitamin B₁₂; about 450 mg to about 550 mg calcium; about 180 IU to about 220 IU vitamin D₃; about 45 mg to about 55 mg magnesium; and about 0.8 mg to about 1.2 mg boron.

In another embodiment of the present invention, the methods may utilize compositions comprising one or more of about 10 mg vitamin B₆; about 1.6 mg folic acid; about 25 μg vitamin B₁₂; about 500 mg calcium; about 200 IU vitamin D₃; about 50 mg magnesium; and about 1 mg boron.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that may become apparent to those skilled in the art from this detailed description.

### DETAILED DESCRIPTION OF THE INVENTION

It is understood that the present invention is not limited to the particular methodologies, protocols, fillers, and excipients, etc. . . . , described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a vitamin” is a reference to one or more vitamins and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “subject,” as used herein, comprises any and all organisms and includes the term “patient.” “Subject” may refer to a human or any other animal.

The phrase “pharmaceutically acceptable,” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “swallowable form” refers to any compositions that do not readily dissolve when placed in the mouth and may be swallowed whole without any chewing or discomfort. Such compositions, in one embodiment, may have a shape containing no sharp edges and a smooth, uniform and substantially bubble free outer coating.

The phrase “chewable form” refers to any relatively soft compositions that are chewed in the mouth after oral administration, have a pleasant taste and mouthfeel, and quickly break into smaller pieces and begin to dissolve after chewing such that they can be swallowed substantially as a solution.

The phrase “dissolvable form” refers to any compositions that dissolve into a solution in the mouth. Such compositions, in one embodiment, may dissolve within about 60 seconds or less after placement in the mouth without any chewing.

The term “mouthfeel” refers to non-taste-related aspects of the pleasantness experienced by a person while chewing or swallowing a nutritional supplement. Aspects of mouthfeel include, for example and without limitation, the hardness and brittleness of a composition, whether the composition is chewy, gritty, oily, creamy, watery, sticky, easily dissolved, astringent, effervescent, and the like, and the size, shape, and form of the composition (tablet, powder, gel, etc. . . . ).

As stated earlier, cardiovascular disease is the number one cause of death for adults in the United States. Colorectal cancer is the second leading cause of death from cancer in the United States, claiming approximately 55,000 lives each year. Further, an estimated 28 million Americans suffer from osteoporosis. Recent developments in nutritional research suggest that nutritional supplementation with certain vitamins and minerals, as an adjunct to proper diet, exercise and medical care, can aid in preventing, treating and/or alleviating the occurrence or negative effects of these diseases.

The compositions and methods of the present invention provide means to optimize good health by utilizing vitamin and mineral combinations that specifically aim to prevent, treat and/or alleviate the occurrence or negative effects of cardiovascular disease, colorectal cancer and osteoporosis. The compositions and methods of the present invention may be administered to or directed to a subject such as a human or any other organism. Each of the added vitamins and minerals that can be included in the present invention, including B-complex vitamins, such as vitamins B₆, B₉ and/or B₁₂, calcium, vitamin D₃, magnesium and boron, plays a specific role in preventing, treating and/or alleviating the occurrence or negative effects of cardiovascular disease, colorectal cancer and/or osteoporosis. In a specific embodiment, vitamins and minerals that inhibit these compounds' beneficial effects, including vitamin A, vitamin K, iron and lactose, may be specifically excluded from the compositions and methods of the present invention. Further, in another specific embodiment, other added vitamins and/or minerals can be excluded. For example, the compositions and methods of the present invention may be substantially free of added beta carotene; substantially free of added alpha carotene; substantially free of added lutein; substantially free of added lycopene; substantially free of added zeaxanthin; substantially free of added vitamin B₁; substantially free of added vitamin B₂; substantially free of added vitamin B₃; substantially free of added vitamin B₄; substantially free of added vitamin B₅; substantially free of added vitamin B₆; substantially free of added vitamin B₇; substantially free of added vitamin B₈; substantially free of added vitamin B₉; substantially free of added vitamin B₁₀; substantially free of added vitamin B₁₁; substantially free of added vitamin B₁₂; substantially free of added vitamin C; substantially free of added vitamin D₃; substantially free of added vitamin E; substantially free of added calcium; substantially free of added chromium; substantially free of added copper; substantially free of added magnesium; substantially free of added manganese; substantially free of added selenium; substantially free of added zinc; substantially free of added boron; substantially free of added odorless garlic; substantially free of added coenzyme Q-10; substantially free of added 1-carnitine; substantially free of added grape seed extract; substantially free of added green tea extract; substantially free of added quercetin; substantially free of added hawthorne berries; and/or substantially free of added alpha lipoic acid.

B-complex vitamins are water-soluble nutrients that generally are not stored in the body. These vitamins play a variety of roles within the body. They may be included in the compositions and methods of the present invention due to their roles in preventing, treating and/or alleviating the occurrence or negative effects of cardiovascular disease and colorectal cancer. The B-complex vitamins that may be included in the compositions and methods of the present invention comprise one or more of vitamin B₆, vitamin B₉, and vitamin B₁₂.

B-complex vitamins help prevent, treat and/or alleviate the occurrence or negative effects of cardiovascular disease through their involvement in the metabolism and breakdown of homocysteine. Homocysteine is an intermediary product produced by metabolism of the amino acid methionine. Elevated levels of homocysteine have been correlated to an increased risk of cardiovascular disease. Maxwell, Suppl 1 BASIC RES. CARDIOL. 165-71 (2003). Elevated levels of homocysteine may lead to increased risk of cardiovascular disease due to this metabolite's numerous deleterious effects on the vascular system including impairing endothelial function, inducing thrombosis, and increasing oxidant stress. Schlaich, 153(2) ATHEROSCLER. 383-89 (2000); Hanratty, 85(3) HEART 326-30 (2001). Efficient breakdown of this intermediary product is necessary to avoid these deleterious effects on the vascular system, and the metabolic breakdown pathways of homocysteine require vitamins B₆, B₉ and B₁₂. Thus, optimizing the levels of these vitamins has cardio-protective effects by promoting the efficient degradation of homocysteine. Haynes, 16(5) CARDIOVASC. DRUGS THER. 391-9 (2002).

Vitamin B₉ also may have additional physiological effects beyond its role in homocysteine breakdown that protect against cardiovascular disease. Bailey, 133(6) J. NUTR. 1961S-68S (2003); Doshi, 41(11) CLIN. CHEM. LAB. MED. 1505-12 (2003); Haynes, supra. For example, vitamin B₉ improves the levels and functioning of the health promoting, endothelial-derived compound nitric oxide (NO). Das, 19(7-8) NUTR. 686-92 (2003). Vitamin B₉ creates this effect by enhancing the activity of the enzyme nitric oxide synthase, stimulating endogenous tetrahydrobiopterin, and inhibiting generation of intracellular superoxide. All of these actions enhance the half-life of NO thus creating cardioprotective effects. Lucock, 71 MOL. GENET. METAB. 121-38 (2000).

In addition to its cardioprotective effects, vitamin B₉ supplementation and resulting improved folate status also decreases the risk of developing cancers in selected tissues—most notably the colorectum. Bailey, supra; Young—In, 57 NUTR. REVIEWS 314-24 (1999); Giovanucci, 129 ANN. INTERN. MED. 517-24 (1998). Vitamin B₉ supplementation may protect against colorectal and other cancers as a result of its central role in nucleotide synthesis. Specifically, folic acid plays a key role in the formation of nucleic acid precursors such as thymidylic acid and purine nucleotides. A decrease in the formation of these precursors affects the metabolic pathways involved in deoxyribonucleic acid (DNA) methylation, biosynthesis, and stability. Instability in these metabolic pathways can result in aberrant DNA synthesis and repair thus enhancing carcinogenesis by altering the expression of critical tumor suppressor genes and proto-oncogenes. Sergio et al., 3 NATURE REV. CANC. 601-14 (2003); Lucock, supra. Ensuring adequate levels of nucleic acid precursors through Vitamin B₉ supplementation can serve to prevent, treat and/or alleviate the occurrence or negative effects of these cancer-promoting effects.

Nutritional supplementation with the B-complex vitamins B₆, B₉ and B₁₂ can help prevent, treat and/or alleviate the occurrence or negative effects of cardiovascular disease and colorectal cancer. In a specific embodiment, the compositions and methods of the present invention may comprise or use vitamin B₆ in the form of pyridoxine hydrochloride. In another specific embodiment the compositions and methods of the present invention may include vitamin B₆ in amounts ranging from about 5 mg to about 15 mg. In another specific embodiment the compositions and methods of the present invention may include vitamin B₆ in amounts ranging from about 8 mg to about 12 mg. In another specific embodiment the compositions and methods of the present invention may include vitamin B₆ in amounts ranging from about 9 mg to about 11 mg. In another specific embodiment, the compositions and methods of the present invention may include vitamin B₆ in an amount of about 10 mg.

The compositions and methods of the present invention may include vitamin B₉. In a specific embodiment, vitamin B₉ may be included in the form of folic acid. In another specific embodiment, vitamin B₉ may be included in amounts ranging from about 1 mg to about 3 mg. In another specific embodiment, vitamin B₉ may be included in amounts ranging from about 1 mg to about 2.2 mg. In another specific embodiment, vitamin B₉ may be included in amounts ranging from about 1.5 mg to about 1.75 mg. In another embodiment, vitamin B₉ may be included in an amount of about 1.6 mg. In other embodiments of the present invention, vitamin B₉ may be included in one or more of the forms of folacin, metafolin, folate and natural isomers thereof.

Vitamin B₁₂ also may be included in the compositions and methods of the present invention. In one embodiment, vitamin B₁₂ may be included in the form of cyanocobalamin. In another specific embodiment of the present invention, vitamin B₁₂ may be included in amounts ranging from about 12 μg to about 38 μg. In another specific embodiment of the present invention, vitamin B₁₂ may be included in amounts ranging from about 20 μg to about 30 μg. In another specific embodiment of the present invention, vitamin B₁₂ may be included in amounts ranging from about 22 μg to about 28 μg. In another embodiment of the present invention, vitamin B₁₂ may be included in an amount of about 25

The skeletal system is a constantly regenerating matrix of living tissue. It undergoes a process of breaking down and rebuilding, known as remodeling, that is regulated by a group of specialized bone cells called osteoblasts and osteoclasts. Osteoblasts build bone mass by synthesizing collagen, while osteoclasts break down bone through their ability to secrete acids and enzymes in a process called resorption. With this constant change comes a risk of a disturbance to the remodeling process leading to the degenerative disease osteoporosis, or simply a loss of bone mass and density. Such a disturbance can result or be exacerbated by inadequate consumption of nutrients essential for bone health, most notably calcium. Accordingly, calcium supplementation has shown both preventive and therapeutic benefits for osteoporosis. NIH Consensus Development Panel, supra; Shils et al., MODERN NUTRITION IN HEALTH AND DISEASE 141-55 (9^{th} ed. 1999); O'Brien, 56 NUTR. REV. 148-50 (1998); Dowson-Hughes et al., 328 N. ENGL. J. MED. 670-76 (1997); Reid et al., 328 N. ENGL. J. MED. 460-64 (1993).

Calcium is the most abundant mineral in the human body with 99% stored in the bones and teeth. Although the remaining systemic calcium outside of the bones and teeth comprise only 1% of the whole, it is delicately balanced and involved in critical physiological functions. These processes include blood pressure modulation, muscle contraction, nerve transmission and blood clotting. Shils, et al., at 141-55. Recent research also has shed light on the possibility that supplemental calcium may reduce the risk of colorectal cancer. Weingarten, et al., 1 COCHRANE DATABASE SYST REV. CD003548 (2004); Grau et al., 95(23) J. NATL. CANC. INST. 1765-71 (2003); Sergio et al., supra; Baron et al., 340 N. ENG. J. MED. 101-7 (1997). Two primary theories as to this preventive role include calcium's ability to precipitate bile and fatty acids that can stimulate the proliferation of colon cells. A second hypothesis is the effect of calcium on extracellular calcium-sensing receptors with resulting growth inhibition on colon carcinoma cells. Bonner et al., 13(12) ONCOL. RES. 551-59 (2003); Kalley et al., 24 CANCER DETECTION AND PREVENTION 127-36 (2000). The preventive properties of calcium appear most beneficial in those at high risk of colorectal cancer secondary to genetics, lifestyle, or a medical history of colonic polyps.

Due to its ability to prevent, treat and/or alleviate the occurrence or negative effects of osteoporosis and colorectal cancer, the compositions and methods of the present invention may include calcium in either chelated or non-chelated form. In a specific embodiment, calcium may be included in the form of calcium carbonate. In another specific embodiment, calcium may be included in amounts ranging from about 250 mg to about 750 mg. In another specific embodiment, calcium may be included in amounts ranging from about 400 mg to about 600 mg. In another specific embodiment, calcium may be included in amounts ranging from about 450 mg to about 550 mg. In another specific embodiment, calcium may be included in an amount of about 500 mg.

Vitamin D is an essential nutrient with pro-hormone activity that is required for multiple systemic functions. Vitamin D specifically is included in the compositions and methods of the present invention due to its roles in preventing, treating and/or alleviating the occurrence or negative effects of osteoporosis and colorectal cancer. Grau, supra; DeLuca et al., 56 NUTR. REV. S4-S10 (1998).

Vitamin D is a fat-soluble substance important for the maintenance of healthy bones. NIH Consensus Development Panel, supra. Sources of vitamin D include dietary and supplemental sources as well as synthesis in the skin from 7-dehydrocholesterol via photochemical reactions using ultraviolet-B(UV-B) radiation from sunlight. The (UV-B) source of vitamin D makes specific population groups more susceptible to deficiency, notably the elderly, institutionalized, and those in sunlight deficient climates.

Vitamin D acts proactively for bone health by regulating systemic calcium homeostasis. Vitamin D increases calcium and phosphorous absorption from the gastrointestinal tract, improves calcium reabsorption into bone tissue, and has a modulating effect on the parathyroid gland. DeLuca et al., supra. These functions aid in optimizing calcium metabolism and utilization. While vitamin D deficiency alone can produce a deficit in bone mineralization, turnover and loss with resulting osteomalacia (softening of the bones), studies have shown that vitamin D in conjunction with calcium supplementation has preventive and therapeutic benefits for osteoporosis. Shils et al., supra; O'Brien, supra; Dowson-Hughes et al., supra.

Vitamin D also may have preventive properties in regard to colorectal cancer. The proposed preventive mechanism may be in its systemic enhancement of calcium's overall bodily utilization, or secondary to an independent, separate activity. Holt, 11(1) CANC. EPIDEMIOL. BIOMARKERS PREV. 113-19 (2002). These alternate functions include a dose dependant inhibition of colon cancer proliferation, thus switching proliferation to differentiation. Vitamin D also has been shown to be beneficial in protecting against oxidative DNA damage in both normal and malignant colonic tissue. Kallay, 40(8) FOOD CHEM. TOXICOL. 1191-96 (2002).

In a specific embodiment, the novel compositions and methods of the present invention may comprise or use vitamin D₃. In a specific embodiment vitamin D₃ may be in the form of cholecalciferol. In another specific embodiment, the compositions and methods of the present invention may include vitamin D₃ in amounts ranging from about 100 IU to about 300 IU. In another specific embodiment, the compositions and methods of the present invention may include vitamin D₃ in amounts ranging from about 160 IU to about 240 IU. In another specific embodiment, the compositions and methods of the present invention may include vitamin D₃ in amounts ranging from about 180 IU to about 220 IU. In another specific embodiment, the compositions and methods of the present invention may include vitamin D₃ in an amount of about 200 IU.

Magnesium also plays a variety of roles within the body. Magnesium is specifically included in the compositions and methods of the present invention due to its role in preventing, treating and/or alleviating the occurrence or negative effects of cardiovascular disease and osteoporosis.

Magnesium deficiency may be related to cardiovascular disease and hypertension due in part to its role as an important modulator of calcium and potassium channels in cardiac muscle. Specifically, magnesium is critical for the maintenance of electrochemical potentials of nerve and muscle membranes and the neuromuscular junction transmissions, particularly important in the heart. Iseri, 108 AM. HEART J. 188-93 (1984). Not surprisingly then, magnesium deficiency is tied to cardiovascular disease and hypertension. Agus et al., 17 CRIT. CARE CLIN. 175-87 (2001). Indeed, oral magnesium therapy improves endothelial function in patients with coronary disease. Shechter et al., 102 CIRCULATION 2353-58 (2000).

Magnesium plays a key role in bone mineralization. Dima et al., 83(8) J. ENDOCRIN. MET. 2742-48 (1998). It is essential in activating bone building osteoblasts and enhancing the sensitivity of bone tissue to parathyroid hormone. Magnesium also plays a key role in the optimal utilization of vitamin D. Shils et al., supra. Accordingly, studies have demonstrated an increase of bone mineral density in postmenopausal women which was associated with their intake of supplemental magnesium.

The novel compositions and methods of the present invention may comprise or use magnesium, in either chelated or non-chelated form. In a specific embodiment, magnesium may be included in the compositions and methods of the present invention in the form of magnesium oxide. In another embodiment of the present invention, magnesium may be included in amounts ranging from about 25 mg to about 75 mg. In another embodiment of the present invention, magnesium may be included in amounts ranging from about 40 mg to about 60 mg. In another embodiment of the present invention, magnesium may be included in amounts ranging from about 45 mg to about 55 mg. In another specific embodiment, magnesium may be included in the amount of about 50 mg.

Boron is a trace nutrient essential for the optimal utilization of calcium, vitamin D and magnesium. Studies indicate that supplemental boron increases the level of the vitamin D metabolite, 25-hydroxycholecalciferol. The introduction of boron also has been shown to counteract the loss of both calcium and magnesium, thus decreasing bone demineralization in postmenopausal women. Proceedings of the 2^{nd} International Symposium on the Health Effects of Boron and its Compounds, 66 BIOL. TRACE ELEM. RES. 1-473 (1998); Nielson et al., 1 FASEB J. 394-97 (1987). In a particular embodiment, the novel compositions and methods of the present invention may comprise or use boron. In one embodiment of the present invention, boron may be included in the form of boron amino acid chelate. In another embodiment, boron may be included in amounts ranging from about 0.5 mg to about 2 mg. In another embodiment, boron may be included in amounts ranging from about 0.5 mg to about 1.5 mg. In another embodiment, boron may be included in amounts ranging from about 0.8 mg to about 1.2 mg. In another embodiment, boron may be included in an amount of about 1 mg.

The compositions and methods of the present invention may comprise or use a combination of the included vitamins and minerals just described in either chelated or non-chelated form. The active ingredients are available from numerous commercial sources, and in several active forms or salts thereof, known to those of ordinary skill in the art. Hence, the compositions and methods of the present invention are not limited to comprising or using any particular form of the vitamin or mineral ingredient described herein.

Nutrition is a constantly evolving health science. Nearly as proliferative as research findings correlating nutrients and disease prevention are findings demonstrating that supplementation with some nutrients can be counter-productive to the health needs of specific populations.

Elevated serum levels of the active form of vitamin A (retinol) are correlated with increased bone fragility with a resulting deleterious effect on bone health. Although retinol is involved in bone remodeling, excessive intake, as can occur with long term supplementation, has been linked to bone demineralization. Michaelson et al., 348(4) N. ENG. J. MED. 287-94 (2003); Feskanich et al., 287(1) JAMA 47-54 (2002). In a specific embodiment, the compositions and methods of the present invention may be free from added vitamin A.

Although iron is an essential nutrient with numerous functions, broad spectrum supplementation among the populous has come under scrutiny due to its role as a catalyst for oxidative stress. Day et al., 107(20) CIRCULATION 2601-06 (2003). Oxidation, notably of Low-Density Lipoprotein (LDL) cholesterol, has been strongly correlated with an increased risk of cardiovascular disease. De Valk et al., 159 ARCH. INT. MED. 1542-48 (1999). Accordingly, iron supplementation is indicated only in specific diagnostic states. In a specific embodiment, the compositions and methods of the present invention may be free from added iron.

Although vitamin K, or phylloquinone, plays a role in the process of maintaining bone health, it also plays a major role in the synthesis of coagulation factors. This delicate balance of coagulation is at times purposefully altered in those with, or at high risk of, cardiovascular disease. Increased intake of vitamin K can alter the efficacy of specific medications used for this purpose. Further, the human body produces vitamin K from naturally occurring intestinal bacteria, thus making deficiency of this nutrient rare. Due to these factors, broad spectrum vitamin K supplementation is discouraged. Kurnik et al., 37(11) ANN. PHARMACOTHER. 1603-06 (2003); Shearer, 345 LANCET 229-34 (1995). In a specific embodiment, the compositions and methods of the present invention may be free from added vitamin K.

Lactose is a disaccharide, or sugar that is found mainly in milk and dairy products. Lactose intolerance or the inability to properly digest and absorb this compound is relatively common. With this inability comes uncomfortable side effects such as abdominal bloating, pain, and diarrhea upon ingestion of lactose-containing foods. Since milk and dairy products are a primary source of both calcium and lactose, those who are lactose intolerant are more likely to have insufficient calcium intake and therefore osteoporosis. DiStefano et al., 122(7) GASTROENTEROL. 1793-99 (2002). In a specific embodiment, the compositions and methods of the present invention may be free of added lactose.

A specific embodiment of the present invention may comprise swallowable compositions. Swallowable compositions are well known in the art and are those that do not readily dissolve when placed in the mouth and may be swallowed whole without any chewing or discomfort. In a specific embodiment of the present invention the swallowable compositions may have a shape containing no sharp edges and a smooth, uniform and substantially bubble free outer coating.

To prepare the swallowable compositions of the present invention, each of the active ingredients may be combined in intimate admixture with a suitable carrier according to conventional compounding techniques. In a specific embodiment of swallowable compositions of the present invention, the surface of the compositions may be coated with a polymeric film. Such a film coating has several beneficial effects. First, it reduces the adhesion of the compositions to the inner surface of the mouth, thereby increasing the patient's ability to swallow the compositions. Second, the film may aid in masking the unpleasant taste of certain drugs. Third, the film coating may protect the compositions of the present invention from atmospheric degradation. Polymeric films that may be used in preparing the swallowable compositions of the present invention include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia. Pharmaceutical carriers and formulations for swallowable compounds are well known to those of ordinary skill in the art. See generally, e.g., WADE & WALLER, HANDBOOK OF PHARMACEUTICAL EXCIPIENTS (2^{nd} ed. 1994).

In a specific embodiment of the present invention, the compositions may comprise chewable compositions. Chewable compositions are those that have a palatable taste and mouthfeel, are relatively soft, and quickly break into smaller pieces and begin to dissolve after chewing such that they are swallowed substantially as a solution.

In order to create chewable compositions, certain ingredients should be included to achieve the attributes just described. For example, chewable compositions should include ingredients that create a pleasant flavor and mouthfeel and promote relative softness and dissolvability in the mouth. The following discussion describes ingredients that may help to achieve these characteristics.

Chewable compositions preferably have a pleasant or palatable flavor. Palatable flavors may be achieved by including sweetening agents and/or flavorants. Sweetening agents that may be included in the compositions of the present invention include, by way of example and without limitation, sucrose, fructose, high fructose corn syrup, dextrose, saccharin sodium, maltodextrin, aspartame, potassium acesulfame, neohesperidin dihydrochalcone, sucralose, monoammonium glycyrrhizinate, and others known to those of ordinary skill in the art. As used herein, the term “flavorant” means natural or artificial compounds used to impart a pleasant flavor and often odor to a pharmaceutical preparation. Flavorants that may be used in the present invention include, for example and without limitation, natural and synthetic flavor oils, flavoring aromatics, extracts from plants, leaves, flowers, and fruits, and combinations thereof. Such flavorants include, by way of example and without limitation, anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leave oil, oil of nutmeg, oil of sage, oil of bitter almonds, cassia oil; citrus oils such as lemon, orange, lime and grapefruit oils; and fruit essences, including apple, pear, peach, berry, wildberry, date, blueberry, kiwi, strawberry, raspberry, cherry, plum, pineapple, and apricot. All of these flavorants are commercially available. In a specific embodiment of the present invention, flavorants that may be used include natural berry extracts and natural mixed berry flavor, as well as citric and malic acid. The amount of flavorants used may depend on a number of factors, including desired taste characteristics. While not necessary, one or more of these sweetening agents and/or flavorants also may be included in the swallowable compositions of the present invention.

In addition to having a palatable flavor, chewable compositions also should have a pleasant mouthfeel. A variety of ingredients can be included in the compositions of the present invention to enhance mouthfeel.

In the chewable compositions of the present invention, sugars such as white sugar, corn syrup, sorbitol (solution), maltitol (syrup), oligosaccharide, isomaltooligosaccharide, sucrose, fructose, glucose, lycasin, xylitol, lactitol, erythritol, mannitol, isomaltose, dextrose, polydextrose, dextrin, compressible cellulose, compressible honey, compressible molasses and mixtures thereof may be added to improve mouthfeel and palatability. Further, by way of example and without limitation, fondant or gums such as gelatin, agar, arabic gum, guar gum, and carrageenan may be added to improve the chewiness of the compositions. Fatty materials also may be included to improve mouthfeel and palatability. Fatty materials that may be included in the present invention include, by way of example and without limitation, vegetable oils (including palm oil, palm hydrogenated oil, corn germ hydrogenated oil, castor hydrogenated oil, cotton-seed oil, olive oil, peanut oil, palm olein oil, and palm stearin oil), animal oils (including refined oil and refined lard whose melting point ranges from 30° to 42° C.), Cacao fat, margarine, butter, and shortening.

Alkyl polysiloxanes (commercially available polymers sold in a variety of molecular weight ranges and with a variety of different substitution patterns) also may be used in the present invention to enhance the texture, the mouth feel, or both of the chewable nutritional supplement compositions described herein. By “enhance the texture” it is meant that the alkyl polysiloxane improves one or more of the stiffness, the brittleness, and the chewiness of the chewable supplement, relative to the same preparation lacking the polysiloxane. By “enhance the mouth feel” it is meant that the alkyl polysiloxane reduces the gritty texture of the supplement once it has liquefied in the mouth, relative to the same preparation lacking the polysiloxane.

Alkyl polysiloxanes generally comprise a silicon and oxygen-containing polymeric backbone with one or more alkyl groups pending from the silicon atoms of the back bone. Depending upon their grade, they can further comprise silica gel. Alkyl polysiloxanes are generally viscous oils. Exemplary alkyl polysiloxanes that can be used in the swallowable, chewable or dissolvable compositions of the present invention include, by way of example and without limitation, monoalkyl or dialkyl polysiloxanes, wherein the alkyl group is independently selected at each occurrence from a C₁-C₆-alkyl group optionally substituted with a phenyl group. A specific alkyl polysiloxane that may be used is dimethyl polysiloxane (generally referred to as simethicone). More specifically, a granular simethicone preparation designated simethicone GS may be used. Simethicone GS is a preparation which contains 30% simethicone USP. Simethicone USP contains not less than about 90.5% by weight (CH₃)₃—Si{OSi(CH₃)₂}CH₃ in admixture with about 4.0% to about 7.0% by weight SiO₂.

To prevent the stickiness that can appear in conventional chewable compositions and to facilitate conversion of the active ingredients to emulsion or suspension upon taking, the compositions of the present invention, may further comprise emulsifiers such as, by way of example and without limitation, glycerin fatty acid ester, sorbitan monostearate, sucrose fatty acid ester, lecithin and mixtures thereof. In a specific embodiment, one or more of such emulsifiers may be present in an amount of about 0.01% to about 5.0%, by weight of the administered compositions. If the level of emulsifier is lower or higher than the said range, the emulsification cannot be realized, or wax value will rise.

Chewable compositions should begin to break and dissolve in the mouth shortly after chewing begins such that the compositions can be swallowed substantially as a solution. The dissolution profile of chewable compositions may be enhanced by including rapidly water-soluble fillers and excipients. Rapidly water-soluble fillers and excipients preferably dissolve within about 60 seconds of being wetted with saliva. Indeed, it is contemplated that if enough water-soluble excipients are included in the compositions of the present invention, they may become dissolvable rather than chewable composition forms. Examples of rapidly water soluble fillers suitable for use with the present invention include by way of example and without limitation, saccharides, amino acids, and the like. In a specific embodiment, the saccharide may be a mono-, di- or oligosaccharide. Examples of saccharides which may be added to the compositions of the invention include, by way of example and without limitation, sorbitol, glucose, dextrose, fructose, maltose and xylitol (all monosaccharides); and sucrose, glucose, galactose and mannitol (all disaccharides). Other suitable saccharides are oligosaccharides. Examples of oligosaccharides are dextrates and maltodextrins. Other water soluble excipients that may be used with the present invention include by way of example and without limitation amino acids such as alanine, arginine, aspartic acid, asparagine, cysteine, glutamic acid, glutamine, glycine, histidine, isoleucine, leucine, lysine, methionine, phenylalanine, proline, serine, threonine, tryptophan, tyrosine and valine.

Disintegrants also may be included in the compositions of the present invention in order to facilitate dissolution. Disintegrants, including permeabilising and wicking agents, are capable of drawing water or saliva up into the compositions which promotes dissolution from the inside as well as the outside of the compositions. Such disintegrants, permeabilising and/or wicking agents that may be used in the present invention include by way of example and without limitation, starches such as corn starch, potato starch, pre-gelatinized and modified starches thereof, cellulosic agents such as Ac-di-sol, montmorrilonite clays, cross-linked PVP, sweeteners, bentonite, microcrystalline cellulose, croscarmellose sodium, alginates, sodium starch glycolate, gums such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents such as hydroxymethylcellulose, hydroxypropylcellulose and hydroxyopropylmethylcellulose.

Finally, dissolution of the compositions may be facilitated by including relatively small particles sizes of the ingredients used.

In addition to those described above, any appropriate fillers and excipients may be utilized in preparing the swallowable, chewable and/or dissolvable compositions of the present invention so long as they are consistent with the objectives described herein. For example, binders are substances used to cause adhesion of powder particles in granulations. Such compounds appropriate for use in the present invention include, by way of example and without limitation, acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium, and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, polyethylene glycol, and others known to those of ordinary skill in the art.

Diluents also may be included in the compositions of the present invention in order to enhance the granulation of the compositions. Diluents can include, by way of example and without limitation, microcrystalline cellulose, sucrose, dicalcium phosphate, starches, and polyols of less than 13 carbon atoms, such as mannitol, xylitol, sorbitol, maltitol, and pharmaceutically acceptable amino acids, such as glycin, and their mixtures.

Lubricants are substances used in composition formulations that reduce friction during composition compression. Lubricants that may be used in the present invention include, by way of example and without limitation, stearic acid, calcium stearate, magnesium stearate, zinc stearate, talc, mineral and vegetable oils, benzoic acid, poly(ethylene glycol), glyceryl behenate, stearyl fumarate, and others known to those of ordinary skill in the art.

Glidants improve the flow of powder blends during manufacturing and minimize composition weight variation. Glidants that may be used in the present invention include by way of example and without limitation, silicon dioxide, colloidal or fumed silica, magnesium stearate, calcium stearate, stearic acid, cornstarch, talc and others known to those of ordinary skill in the art.

Colorants also may be included in the nutritional supplement compositions of the present invention. As used herein, the term “colorant” includes compounds used to impart color to pharmaceutical preparations. Such compounds include, by way of example and without limitation, FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide, red and others known to those of ordinary skill in the art. Coloring agents also can include pigments, dyes, tints, titanium dioxide, natural coloring agents such as grape skin extract, beet red powder, beta carotene, annato, carmine, turmeric, paprika, and others known to those of ordinary skill in the art. It is recognized that no colorant is required in the nutritional supplement compositions described herein.

If desired, compositions may be sugar coated or enteric coated by standard techniques. The unit dose forms may be individually wrapped, packaged as multiple units on paper strips or in vials of any size, without limitation. The swallowable, chewable or dissolvable compositions of the invention may be packaged in unit dose, rolls, bulk bottles, blister packs and combinations thereof, without limitation.

The swallowable, chewable or dissolvable compositions of the present invention may be prepared using conventional methods and materials known in the pharmaceutical art. For example, U.S. Pat. Nos. 5,215,754 and 4,374,082 relate to methods for preparing swallowable compositions. U.S. Pat. No. 6,495,177 relates to methods to prepare chewable nutritional supplements with improved mouth feel. U.S. Pat. No. 5,965,162 relates to compositions and methods for preparing multi-vitamin comestible units which disintegrate quickly in the mouth, especially when chewed. Further, all pharmaceutical carriers and formulations described herein are well known to those of ordinary skill in the art, and determination of workable proportions in any particular instance will generally be within the capability of the person skilled in the art. Details concerning any of the excipients of the invention may be found in WADE & WALLER, supra. All active ingredients, fillers and excipients are commercially available from companies such as Aldrich Chemical Co., FMC Corp, Bayer, BASF, Alexi Fres, Witco, Mallinckrodt, Rhodia, ISP, and others.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

### EXAMPLES

Without further elaboration, it is believed that one skilled in the art, using the preceding description, can utilize the present invention to the fullest extent. The following examples are illustrative only, and not limiting of the remainder of the disclosure in any way whatsoever.

### Example 1

A composition of the following formulation was prepared in chewable form:

| | | |
|---|---|---|
| Vitamin B₆ (pyridoxine hydrochloride) | 10 | mg |
| Vitamin B₉ (folic acid) | 1.6 | mg |
| Vitamin B₁₂ (cyanocobalamin) | 25 | μg |
| Vitamin D (cholecalciferol) | 200 | IU |
| Calcium (calcium carbonate) | 500 | mg |
| Magnesium (magnesium oxide) | 50 | mg |
| Boron (boron amino acid chelate) | 1 | mg |

### Example 2

A study is undertaken to evaluate the effectiveness of the compositions of the present invention in the treatment of patients. The objective of the study is to determine whether oral intake of the compositions results in an improvement of the nutritional status with regard to specific vitamins and minerals contained in the administered compositions.

A double-blind, placebo controlled study is conducted over a six-month period. A total of 120 subjects, aged 30-45 years, are chosen for the study. An initial assessment of the nutritional status of each subject is conducted. Vitamin B₆ is measured by a radioenzymatic assay method wherein serum is incubated with apoenzyme tyrosine-decarboxylase, C₁₄ labeled tyrosine is added to start the enzymatic reaction which is stopped with HCl. Subsequently the free C₁₄-labelled CO₂ is adsorbed by a KOH impregnated filtering paper. The measured C₁₄ activity is directly proportional to the B₆ concentration. Vitamins B₁₂ and folate are measured by quantitative radioassay methods using purified intrinsic factor and purified folate binding protein. Vitamin D is measured using an extraction double-antibody radioimmunoassay (DiaSorin, Inc., Stillwater, Minn.) with a sensitivity of 1.5 ng/ml and intra- and interassay coefficients of variation of 9-13% and 8-11% respectively. Calcium and magnesium are measured using spectrophotometry. Boron is measured using inductively-coupled plasma-mass spectrometry (ICPMS) with an internal standard of ¹⁰B at a final concentration of 50 μg/l.

The 120 subjects are separated into 4 separate groups of 30 subjects. In a first group comprising men, and in a second group comprising women, each subject is administered one dosage form of the composition as described in Example 1 twice a day. In a third group comprising men and a fourth group comprising women, each subject is administered one placebo dosage form twice a day. Thus, dosage form administration occurs every 12 hours. No other nutritional supplements are taken by the subjects during the assessment period.

An assessment of the nutritional status of each subject is conducted utilizing methods described above at one month intervals for a six month period. The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 1, 2, 3, 4, 5 and 6 months, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

A statistically significant improvement in the nutritional status of all vitamin and mineral levels measured is observed in the treated subjects over the controls upon completion of the study. Therefore, the study confirms that oral administration of the compositions of the present invention is effective in improving the nutritional status of patients.

While specific embodiments of the present invention have been described, other and further modifications and changes may be made without departing from the spirit of the invention. All further and other modifications and changes are included that come within the scope of the invention as set forth in the claims. The disclosure of all publications cited above are expressly incorporated by reference in their entireties to the same extent as if each were incorporated by reference individually.

1-112. (canceled)

113. A composition consisting of about 10 mg vitamin B₆, about 1.6 mg vitamin B₉, about 25 μg vitamin B₁₂, about 500 mg calcium, about 200 IU vitamin D₃, about 50 mg magnesium, about 1 mg boron, and one or more pharmaceutically acceptable carriers, wherein said composition is in a chewable chocolate form.

114. The composition of claim 113, wherein said vitamin B₆ comprises pyridoxine.

115. The composition of claim 113, wherein said vitamin B₉ comprises folic acid.

116. The composition of claim 113, wherein said vitamin B₉ comprises folacin.

117. The composition of claim 113, wherein said vitamin B₉ comprises metafolin.

118. The composition of claim 113, wherein said vitamin B₉ comprises folate.

119. The composition of claim 113, wherein said vitamin B₉ comprises natural isomers of folate selected from one or more of the group consisting of (6S)-tetrahydrofolic acid, 5-methyl-(6S)-tetrahydrofolic acid, 5-formyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid or polyglutamyl derivatives thereof.

120. The composition of claim 113, wherein said vitamin B₁₂ comprises cyanocobalamin.

121. The composition of claim 113, wherein said calcium comprises calcium carbonate.

122. The composition of claim 113, wherein said vitamin D₃ comprises cholecalciferol.

123. The composition of claim 113, wherein said magnesium comprises magnesium oxide.

124. The composition of claim 113, wherein said boron comprises boron amino acid chelate.

125. The composition of claim 113, wherein said composition is free of added lactose.

126. The composition of claim 113, wherein said pharmaceutically acceptable carriers are selected from the group consisting of one or more of binders, diluents, lubricants, glidants, colorants, emulsifiers, disintegrants, starches, water, oils, alcohols, preservatives and sugars.

127. A method comprising administering the composition of claim 113 to a patient.
